(19) United States
(12) Patent Application Publication
(10) Pub. No.: US 2024/0115401 A1
(43) Pub. Date: Apr. 11, 2024

(54) ARTIFICIAL BREAST AND SILICONE RUBBER BODY

(71) Applicant: Natural Breast Co,. Ltd., Fukuoka (JP)

(72) Inventor: Yukie HONDA, Fukuoka (JP)

(73) Assignee: Natural Breast Co,. Ltd., Fukuoka (JP)

(21) Appl. No.: 18/276,879

(22) PCT Filed: Feb. 15, 2021
(86) PCT No.: PCT/JP2021/005477

§ 371 Date: Aug. 10, 2023

(51) Int. Cl. A61F 2/52

(52) U.S. Cl. CPC A61F 2/52; A61F 2002/523; A61F 2002/526

(57) ABSTRACT

An artificial beast and a silicone rubber body are provided. This artificial breast comprises a first layer, a second layer, a third layer, a fourth layer, and an outer cover layer. The first layer contains a silicone gel, and the second layer is a harder layer than the first layer, is a part on the outside of the first layer. The third layer is disposed outside the first layer and the second layer and is a harder layer than the first layer and the second layer. The outer cover layer is formed from a colored layer, a cover layer, and a reflection suppression layer.

### BACKGROUND

### Technical Field

The present invention relates to an artificial breast and a silicone rubber body. In detail, the present invention relates to an artificial breast and a silicone rubber body for a woman who has lost her breast, for example.

### Description of Related Art

For a woman, a deformation or loss of her breast has a significant psychological impact. As in need of a shape control pad when putting on underwear, etc., her daily activities are hindered and the quality of life is lowered.

Thus, in order to improve the quality of life, a breast reconstruction surgery is performed as a method of returning the deformed or lost breast to as normal a state as possible. However, the breast reconstruction surgery has disadvantages such as an occurrence of infections or skin necrosis.

Meanwhile, there are many women who dislike such disadvantages and do not undergo the breast reconstruction surgery and instead attach an artificial breast which is an artificial object to the skin of the chest portion, and various artificial breasts have been proposed.

For example, in Patent Literature 1, an artificial breast as shown in FIG. 4 is described. That is, an artificial breast 101 described in Patent Literature 1 includes a bag-shaped portion 102 made of silicone rubber, and a gel material 103 enclosed inside the bag-shaped portion 102.

In addition, the bag-shaped portion 102 is configured by a base layer portion 102A molded to have a predetermined thickness, and a breast outer layer portion 102B formed in a state where a convex curved surface on the surface is close to a breast and molded to have a predetermined thickness. In addition, the artificial breast 101 is put on via an adhesive 301 as a medical material so as to serve as a substitute for a breast lost point 100 in a chest portion after a breast is removed.

### CITATION LIST

### Patent Documents

Patent Document 1: Japanese Unexamined Patent Application Publication No. 2013-000332

### SUMMARY

### Technical Problems

However, the artificial breast described in Patent Literature 1 is configured by a single silicone rubber layer. In a case where the artificial breast is deformed by a force applied from an exterior, unnatural wrinkles are easily made and the artificial breast easily becomes unnaturally hard in comparison to a human breast.

The present invention is made in consideration of the point described above, and an object of the present invention is to provide an artificial breast that is less likely to become unnatural in comparison to a human breast, and a silicone rubber body that is less likely to become unnatural in comparison to the human skin.

### Solution to Problems

In order to achieve the object described above, an artificial breast of the present invention includes a first layer configured by containing silicone gel, a second layer configured by containing silicone rubber and arranged partly on the outer side of the first layer, the second layer being harder than the first layer, a third layer configured by containing silicone rubber and arranged on the outer side of the first layer and the second layer, the third layer being harder than the first layer and the second layer, and a fourth layer configured by containing silicone rubber and arranged partly on the outer side of the third layer, the fourth layer having substantially the same hardness as the third layer, wherein a curing speed of the silicone rubber of the third layer is slower than a curing speed of the silicone rubber of the second layer.

With the first layer configured by containing silicone gel, it is possible to keep softness for a long time in comparison to an artificial breast not configured by containing silicone gel.

In addition, with the second layer arranged partly on the outer side of the first layer, the second layer being harder than the first layer, and the third layer arranged on the outer side of the first layer and the second layer, the third layer being harder than the first layer and the second layer, it is possible to adjust a shape of the artificial breast.

In addition, with the first layer configured by containing silicone gel, the second layer arranged partly on the outer side of the first layer, the second layer being harder than the first layer, and the third layer arranged on the outer side of the first layer and the second layer, the third layer being harder than the first layer and the second layer, the artificial breast becomes harder stepwise from the inner side to the outer side. Thus, even when a force is applied from an exterior, unnatural wrinkles are not easily made.

In addition, with the fourth layer configured by containing silicone rubber and arranged partly on the outer side of the third layer, the fourth layer having substantially the same hardness as the third layer, silicone rubber before curing for forming the fourth layer is arranged on the artificial breast of the present invention whose shape is adjusted by the first to third layers, making it easier to closely attach to an adhesive material.

In addition, in order to arrange the third layer on the outer side of the second layer, the silicone rubber of the third layer is applied to an artificial breast formation mold prior to the silicone rubber of the second layer. Since the curing speed of the silicone rubber of the third layer is slower than the curing speed of the silicone rubber of the second layer, it is possible to make curing of the silicone rubber of the second layer and curing of the silicone rubber of the third layer occur at substantially the same timing. As a result, it is possible to blur a border line between the second layer and the third layer and bring the second layer and the third layer into a nearly integrated state. Thus, it is possible to reduce unnaturalness upon touching the artificial breast of the present invention.

In addition, the artificial breast of the present invention can have a configuration of including a colored layer arranged on the outer side of the third layer, the colored layer being configured by containing a colorant, and a cover layer arranged on the outer side of the colored layer, the cover layer being capable of suppressing peeling of the colored layer.

In this case, with the colored layer arranged on the outer side of the third layer, the colored layer being configured by containing the colorant, it is possible to perform external coloring and bring the texture closer to the texture of the human skin than in the case of internal coloring where the first layer, the second layer, or the third layer contains a colorant.

In addition, with the cover layer arranged on the outer side of the colored layer, the cover layer being capable of suppressing the peeling of the colored layer, the texture brought close to the texture of the human skin is easily maintained.

In addition, the artificial breast of the present invention can have a configuration that the colored layer has a nipple region in which a nipple is displayed, and the nipple region and other regions of the colored layer are positioned in substantially the same plane.

In this case, it is possible to make the nipple inconspicuous even when the nipple is seen through clothing.

In addition, the artificial breast of the present invention can have a configuration of including a reflection suppression layer arranged on the outer side of the cover layer, the reflection suppression layer being capable of suppressing reflection of light of the cover layer or the colored layer.

In this case, it is possible to bring the texture even closer to the texture of the human skin.

In addition, the artificial breast of the present invention can have a configuration of including a thin film layer arranged on the outer side of the fourth layer, the thin film layer being capable of blocking a substance, with the thin film layer being transparent.

In this case, the thin film layer can block a substance. Thus, it is possible to prevent a substance contained in the first layer, the second layer, or the third layer from leaking out to the exterior of the artificial breast. In addition, since the thin film layer is transparent, it does not feel strange.

In addition, the artificial breast of the present invention can have a configuration of including an adhesive layer arranged attachably to and detachably from a layer which is arranged on the outermost side, the adhesive layer being configured by containing an adhesive material. In this case, it is possible to improve an adhesive force of the artificial breast with respect to the human body.

In addition, in the artificial breast of the present invention, the first layer can be configured by containing the silicone rubber without any space. In this case, the artificial breast of the present invention can be a filled type artificial breast where there is no need to refill an artificial breast with air unlike a hollow type artificial breast.

In addition, the artificial breast of the present invention can have a configuration that the third layer is arranged to cover the entire first layer. In this case, the artificial breast is more easily manufactured than a configuration that the entire first layer is covered by layers different from each other.

In addition, in order to achieve the object described above, a silicone rubber body of the present invention includes a silicone rubber layer configured by containing silicone rubber, a colored layer arranged on the outer side of the silicone rubber layer, the colored layer being configured by containing a colorant, a cover layer arranged on the outer side of the colored layer, the cover layer being capable of suppressing peeling of the colored layer, and a reflection suppression layer arranged on the outer side of the cover layer, the reflection suppression layer being capable of suppressing reflection of light of the cover layer or the colored layer.

With the silicone rubber layer configured by containing silicone rubber, it is possible to simulate the feel of the human skin.

In addition, with the colored layer arranged on the outer side of the silicone rubber layer, the colored layer being configured by containing the colorant, it is possible to perform external coloring and bring the texture closer to the texture of the human skin than in the case of internal coloring where silicone rubber contains a colorant.

In addition, with the cover layer arranged on the outer side of the colored layer, the cover layer being capable of suppressing the peeling of the colored layer, it is possible to maintain the texture brought close to the texture of the human skin.

In addition, with the reflection suppression layer arranged on the outer side of the cover layer, the reflection suppression layer being capable of suppressing the reflection of light of the cover layer or the colored layer, it is possible to bring the texture even closer to the texture of the human skin.

### Advantageous Effects of Invention

The artificial breast according to the present invention is less likely to become unnatural in comparison to a human breast. The silicone rubber body according to the present invention is less likely to become unnatural in comparison to the human skin.

### BRIEF DESCRIPTION OF THE DRAWINGS

FIG. 1 is a schematic sectional view showing a first embodiment of an artificial breast to which the present invention is applied.

FIG. 2(a) is a schematic plan view of a second embodiment of the artificial breast to which the present invention is applied; and FIG. 2(b) is a schematic partial sectional view of the vicinity of a nipple region when the artificial breast shown in FIG. 2(a) is cut along line A-A.

FIG. 3 is a schematic sectional view of the artificial breast of the first embodiment to which the present invention is applied, the artificial breast being put on a chest portion of a human body.

FIG. 4 is a schematic sectional view of a conventional artificial breast put on a chest portion of a human body.

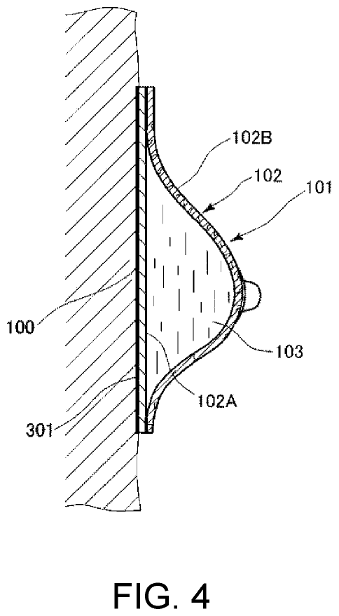

### DESCRIPTION OF THE EMBODIMENTS

Hereinafter, embodiments of the present invention will be described with reference to the attached drawings in order to facilitate understanding of the present invention.

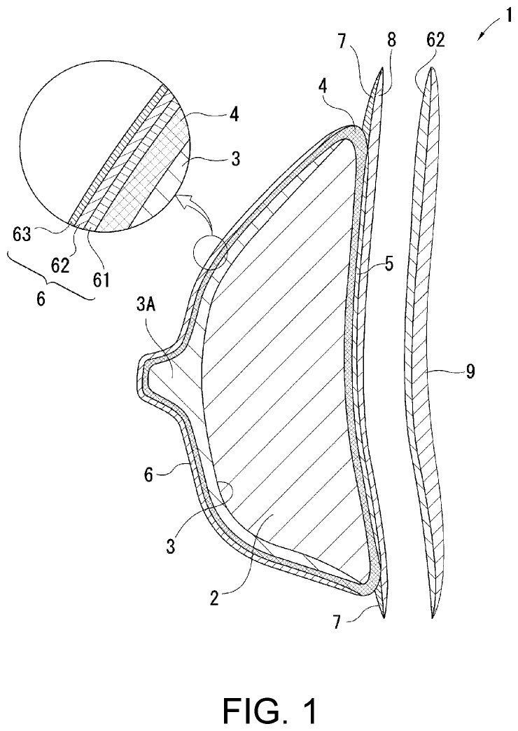

FIG. 1 is a schematic sectional view showing a first embodiment of an artificial breast to which the present invention is applied.

An artificial breast 1 of the present invention shown in FIG. 1 includes a first layer 2. The first layer 2 is configured by containing silicone gel without any space, and is different from a hollow type artificial breast inside which a hollow space is formed. In addition, softness of the first layer 2 having such a configuration is kept at least for 2 years.

In addition, the artificial breast 1 of the present invention includes a second layer 3. The second layer 3 is configured by containing silicone rubber. In addition, the second layer 3 is arranged partly on the outer side of the first layer 2 and is a layer harder than the first layer 2. That is, the second layer 3 does not cover the entire first layer 2 and is laminated on an outer side part of the first layer 2 on one side.

In addition, the second layer 3 is curved, and a protruding nipple portion 3A is integrally provided in part of a convex curved part on one side of the second layer 3. However, it is also possible to attach a nipple portion to the second layer 3 as a separate body.

In addition, as the silicone rubber used for the second layer 3, any silicone rubber can be used as long as the silicone rubber is harder than the silicone gel used for the first layer 2.

In addition, the artificial breast 1 of the present invention includes a third layer 4. The third layer 4 is configured by containing silicone rubber. In addition, the third layer 4 is arranged on the outer side of the first layer 2 and the second layer 3, and is a layer harder than the first layer 2 and the second layer 3. That is, the third layer 4 is directly laminated on the outer side part of the first layer 2 in a state of covering the entire first layer 2 and also laminated on the outer side part of the first layer 2 via the second layer 3.

In addition, a curing speed of the silicone rubber of the third layer 4 is slower than a curing speed of the silicone rubber of the second layer 3.

In order to arrange the third layer 4 on the outer side of the second layer 3, the silicone rubber of the third layer 4 is applied to an artificial breast formation mold prior to the silicone rubber of the second layer 3. Since the curing speed of the silicone rubber of the third layer 4 is slower than the curing speed of the silicone rubber of the second layer 3, it is possible to make curing of the silicone rubber of the second layer and curing of the silicone rubber of the third layer occur at substantially the same timing. As a result, it is possible to blur a border line between the second layer 3 and the third layer 4 and bring the second layer 3 and the third layer 4 into a nearly integrated state. Thus, it is possible to reduce unnaturalness upon touching the artificial breast of the present invention.

In addition, as the silicone rubber used for the third layer 4, any silicone rubber can be used as long as the silicone rubber is harder than the silicone rubber used for the first layer 2 and the silicone rubber used for the second layer 3.

In addition, the artificial breast 1 of the present invention includes a fourth layer 5. The fourth layer 5 is configured by containing silicone rubber. In addition, the fourth layer 5 is arranged partly on the outer side of the third layer 4 and is a layer having substantially the same hardness as the third layer 4. That is, the fourth layer 5 does not cover the entire third layer 4 and is laminated on an outer side part of the third layer 4 on the opposite side of the side where the third layer 4 is in contact with the first layer 2.

In addition, as the silicone rubber used for the fourth layer 5, any silicone rubber can be used as long as the silicone rubber has substantially the same hardness as the silicone rubber used for the third layer 4.

In addition, the artificial breast 1 of the present invention includes an outer covering layer 6. The outer covering layer 6 is arranged on the outer side of the third layer 4. That is, the outer covering layer 6 is laminated on the outer side part of the third layer 4 on the opposite side of the side where the third layer 4 is in contact with the second layer 3.

In addition, the part of the third layer 4 on which the outer covering layer 6 is laminated is a part to be positioned on the front surface side when the artificial breast 1 of the present invention is put on a chest portion of a human body.

In addition, the outer covering layer 6 is configured by a colored layer 61, a cover layer 62, and a reflection suppression layer 63.

The colored layer 61 is configured by containing a colorant, specifically, for example, a pigment. In addition, the colored layer 61 is laminated on the outer side part of the third layer 4 on the opposite side of the side where the third layer 4 is in contact with the second layer 3.

In addition, the cover layer 62 is capable of suppressing peeling of the colored layer 61. In addition, the cover layer 62 is arranged on the outer side of the colored layer 61. That is, the cover layer 62 is laminated on the colored layer 61 on the opposite side of the third layer 4.

In addition, the reflection suppression layer 63 is capable of suppressing reflection of light of the cover layer 62 or the colored layer 61 by roughening a surface of the cover layer 62 or the colored layer 61. In addition, the reflection suppression layer 63 is arranged on the outer side of the cover layer 62. That is, the reflection suppression layer 63 is laminated on the cover layer 62 on the opposite side of the colored layer 61.

In addition, as shown in FIG. 1, the artificial breast 1 of the present invention has a curved part to be positioned on the front surface side when the artificial breast 1 is put on the chest portion of the human body, and a substantially flat part to be positioned on the back surface side facing the chest portion when the artificial breast 1 is put on the chest portion of the human body. A part where the curved part and the flat part are connected is a peripheral edge portion.

In addition, it is also possible to form a concave portion in the artificial breast of the present invention on the back surface side facing the chest portion when the artificial breast is put on the chest portion of the human body.

In addition, the artificial breast 1 of the present invention includes a thin skin portion 7. The thin skin portion 7 is configured by containing silicone rubber, and is so thin that it becomes transparent and is a soft part. In addition, the thin skin portion 7 is provided on the outer side of the third layer 4 over the entire peripheral edge portion. Therefore, when the artificial breast 1 of the present invention is put on the chest portion of the human body, it is possible to see the color of the human skin through the thin skin portion 7.

The thin skin portion 7 is attached to the third layer 4 as a separate body from the third layer 4. However, the thin skin portion 7 may be provided integrally with the third layer 4. In addition, in the example shown in FIG. 1, the thin skin portion 7 is attached to the third layer 4 as a separate body from the fourth layer 5. However, the thin skin portion 7 may be provided in the third layer 4 integrally with the fourth layer 5.

In addition, the artificial breast 1 of the present invention includes a polyethylene sheet layer 8. The polyethylene sheet layer 8 is capable of blocking air, water, a phosphorus substance, etc., and is transparent. In addition, a thickness of the polyethylene sheet layer 8 is specifically, for example, 10 to 20 μm. However, as a matter of course, the present invention is not limited to this thickness. Polyethylene is an example of a material of a thin film layer, and it is possible to use other materials as the thin film layer as long as the materials are capable of blocking a substance and are transparent.

In addition, the polyethylene sheet layer 8 is arranged on the outer side of the fourth layer 5. That is, the polyethylene sheet layer 8 is laminated on a substantially flat part of the fourth layer 5 on the opposite side of the third layer 4. In addition, part of the polyethylene sheet layer 8 is also laminated on the thin skin portion 7. The polyethylene sheet layer 8 is an example of the thin film layer.

In addition, it is also possible to arrange the thin film layer such as the polyethylene sheet layer 8, for example, in a state where the thin film layer is sandwiched between the first layer 2 and the third layer 4 and between the second layer 3 and the third layer 4. In this case, the state is that the thin film layer such as the polyethylene sheet layer covers the entire first layer 2. Thus, it is possible to prevent the silicone gel of the first layer 2 from oozing from a surface of the artificial breast of the present invention to an exterior by the thin film layer such as the polyethylene sheet layer.

In addition, the artificial breast 1 of the present invention includes an adhesive layer 9. The adhesive layer 9 is configured by containing silicone gel, and arranged attachably to and detachably from the polyethylene sheet layer 8 arranged on the outermost side. That is, the adhesive layer 9 is laminated attachably to and detachably from the polyethylene sheet layer 8 via the cover layer 62. However, it is also possible to omit the cover layer 62. In addition, it is also possible to laminate the adhesive layer 9 attachably to and detachably from the polyethylene sheet layer 8 via another cover layer configured by a different material from the material configuring the cover layer 62.

The silicone gel is an example of an adhesive material, and it is also possible to use, for example, a mixture of silicone gel and silicone rubber as the adhesive material.

The artificial breast of the present invention does not necessarily have to include the colored layer, and for example, the first layer, the second layer, or the third layer can also contain a colorant.

However, it is preferable that the artificial breast of the present invention includes the colored layer since it is possible to perform external coloring and bring the texture closer to the texture of the human skin than in the case of internal coloring where the first layer, the second layer, or the third layer contains a colorant.

In addition, the artificial breast of the present invention does not necessarily have to include the cover layer. However, it is preferable that the artificial breast of the present invention includes the cover layer since the texture brought close to the texture of the human skin is easily maintained.

In addition, the artificial breast of the present invention does not necessarily have to include the reflection suppression layer. However, it is preferable that the artificial breast of the present invention includes the cover layer since it is possible to bring the texture even closer to the texture of the human skin.

In addition, the artificial breast of the present invention does not necessarily have to include the polyethylene sheet layer, that is, the thin film layer.

However, it is preferable that the artificial breast of the present invention includes the thin film layer since the thin film layer can block a substance, and thus it is possible to prevent a substance contained in the first layer, the second layer, or the third layer from leaking out to the exterior of the artificial breast. In particular, when a substance such as stratum corneum or phosphorus entering the first layer, the second layer, or the third layer moves to and enters the adhesive layer, an adhesive force of the adhesive layer is decreased. Thus, it is possible to suppress a decrease in the adhesive force of the adhesive layer by the thin film layer.

In addition, the artificial breast of the present invention does not necessarily have to include the adhesive layer as long as the artificial breast of the present invention can be put on the chest portion of the human body. It is also possible to put the artificial breast of the present invention on the chest portion of the human body by using other adhesives. However, it is preferable that the artificial breast of the present invention includes the adhesive layer since it is possible to improve an adhesive force of the artificial breast of the present invention with respect to the human body.

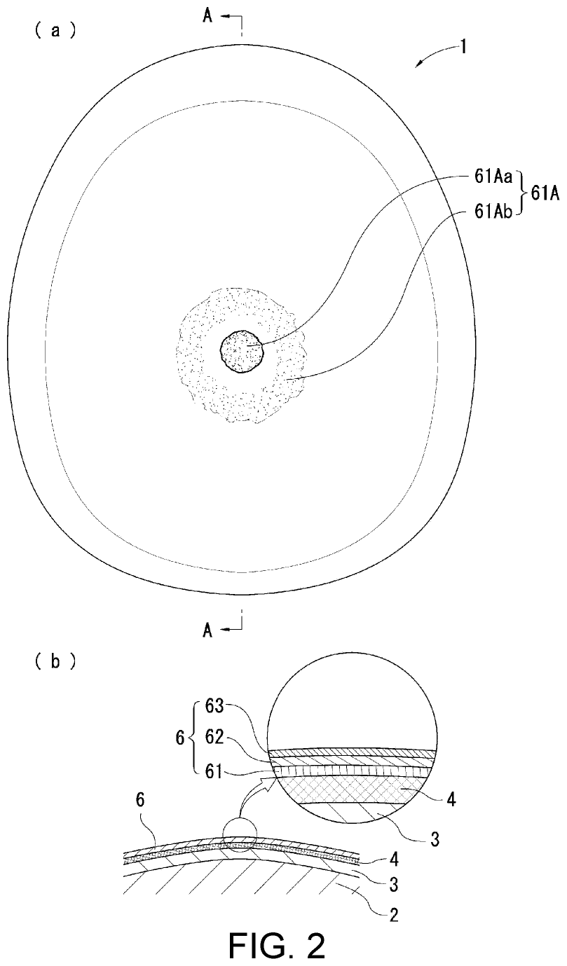

FIG. 2(a) is a schematic plan view of a second embodiment of the artificial breast to which the present invention is applied, and FIG. 2(b) is a schematic partial sectional view of the vicinity of a nipple region when the artificial breast shown in FIG. 2(a) is cut along line A-A.

An artificial breast 1 of the second embodiment of the present invention shown in FIG. 2 is different from the artificial breast 1 of the first embodiment of the present invention shown in FIG. 1 in a point that a nipple portion is not provided in a second layer 3, a colored layer 61 has a nipple region 61A in which a nipple is displayed, and the nipple region 61A and other regions of the colored layer 61 are positioned in substantially the same plane. It is noted that the artificial breast of the second embodiment of the present invention is the same as the artificial breast of the first embodiment of the present invention in other points.

The nipple region 61A is configured by a papilla region 61Aa and an areola region 61Ab shown around the papilla region 61Aa.

As shown in FIG. 2, in the artificial breast 1 of the second embodiment of the present invention, the nipple region 61A and other regions of the colored layer 61 are positioned in substantially the same plane, and the nipple region 61A does not protrude. Thus, by putting on the artificial breast 1 of the second embodiment of the present invention, it is possible to make the nipple inconspicuous even when the nipple is seen through clothing.

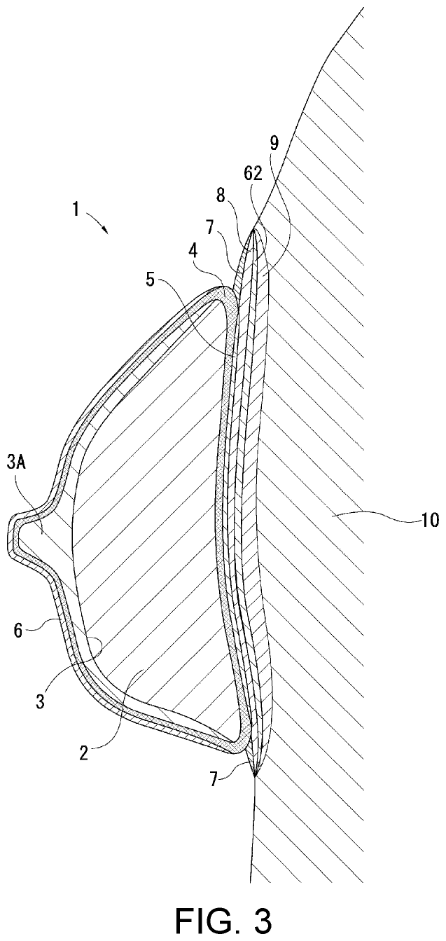

FIG. 3 is a schematic sectional view of the artificial breast of the first embodiment to which the present invention is applied, the artificial breast being put on a chest portion of a human body. As shown in FIG. 3, the artificial breast 1 of the first embodiment of the present invention is put on a chest portion 10 in a state where the adhesive layer 9 is sandwiched between the polyethylene sheet layer 8 and the chest portion 10 of the human body.

In addition, when the artificial breast 1 of the first embodiment of the present invention is detached from the chest portion 10, for example, the thin skin portion 7 of the artificial breast 1 of the present invention is pinched by fingers and the artificial breast 1 of the present invention is pulled apart while slightly compressing the artificial breast 1 of the present invention and letting air in between the adhesive layer 9 and the chest portion 10.

In addition, a silicone rubber body of the present invention is made by laminating the same colored layer, the same cover layer, and the same reflection suppression layer as the artificial breast of the present invention on a silicone rubber layer configured by containing silicone rubber.

### Example 1

A first silicone gel material (trade name: GEL-GUM10(A), manufactured by TOMITA MATEQS) and a second silicone gel material (trade name: GEL-GUM10(B), manufactured by TOMITA MATEQS) were mixed, and a silicone gel mixture was obtained.

In addition, first silicone rubber (trade name: VST-05, manufactured by Factor II, Incorporated) and a curing agent (trade name: VST-05-C, manufactured by Factor II, Incorporated) were mixed, and a first silicone rubber mixture was obtained.

In addition, second silicone rubber (trade name: VST-50, manufactured by Factor II, Incorporated) and a curing agent (trade name: VST-50-C, manufactured by Factor II, Incorporated) were mixed, and a second silicone rubber mixture was obtained.

In order to form the third layer 4 of the artificial breast 1 of the present invention, the second silicone rubber mixture was applied to a first artificial breast formation mold in which a recessed part formed in a shape corresponding to an external form of the artificial breast which includes the nipple portion and a peripheral edge part positioned around the recessed part are provided.

Meanwhile, in order to form the third layer 4 of the artificial breast 1 of the present invention, the second silicone rubber mixture was also applied to a second artificial breast formation mold in which a raised portion is provided. Next, in order to form the second layer 3 of the artificial breast 1 of the present invention, the first silicone rubber mixture was applied to the second silicone rubber mixture applied to the first artificial breast formation mold.

The curing speed of the silicone rubber of the third layer 4 is slower than the curing speed of the silicone rubber of the second layer 3.

Further, in order to form the first layer 2 of the artificial breast 1 of the present invention, the silicone gel mixture was slowly poured into the recessed part of the first artificial breast formation mold to which the first silicone rubber mixture and the second silicone rubber mixture were applied. In addition, an extremely small amount of the silicone gel mixture was also applied to the second silicone rubber mixture applied to the second artificial breast formation mold.

Next, the first artificial breast formation mold and the second artificial breast formation mold were attached to each other while letting the points to which the silicone rubber and the silicone gel were applied face each other.

At this time, no clearance exists between the recessed part of the first artificial breast formation mold and the raised part of the second artificial breast formation mold but the silicone rubber and the silicone gel exist.

After at least 12 hours passed after the close attachment, the first artificial breast formation mold and the second artificial breast formation mold in an attached state were cured within a temperature range from room temperature to 70° C.

After the curing was finished, the second artificial breast formation mold was pulled apart from the first artificial breast formation mold, and a silicone rubber molded product was left in the first artificial breast formation mold.

By a peripheral edge part where the first artificial breast formation mold and the second artificial breast formation mold were attached to each other closely, a thin skin part of the peripheral edge portion of the artificial breast 1 of the present invention was formed. However, after all the thin skin part was cut off, in order to form the thin skin portion 7 of the artificial breast 1 of the present invention, the second silicone rubber mixture was thinly applied to the peripheral edge part of the first artificial breast formation mold.

The first artificial breast formation mold was cured within the temperature range from room temperature to 70° C.

In addition, in order to form the fourth layer 5 of the artificial breast 1 of the present invention, the second silicone rubber mixture was applied to the silicone molded product positioned in the recessed part of the first artificial breast formation mold.

Next, in order to form the polyethylene sheet layer 8, a polyethylene sheet having a thickness of 15 μm was arranged on the second silicone rubber mixture applied to the silicone molded product.

In order to form the cover layer 62, a mixture of the silicone rubber (trade name: VST-50, manufactured by Factor II, Incorporated) and the curing agent (trade name: VST-50-C, manufactured by Factor II, Incorporated) was applied to the polyethylene sheet.

Meanwhile, a first silicone gel material (trade name: GEL-GUM10(A), manufactured by TOMITA MATEQS) and a second silicone gel material (trade name: GEL-GUM10(B), manufactured by TOMITA MATEQS) were mixed, and a silicone gel material mixture was obtained. The obtained silicone gel material mixture and the silicone rubber (trade name: VST-05, manufactured by Factor II, Incorporated) were mixed by a mass ratio of 10:1, and a silicone mixture was obtained.

Next, in order to form the adhesive layer 9, the silicone mixture was applied to the mixture of the silicone rubber and the curing agent applied to the polyethylene sheet, and the first artificial breast formation mold was cured within the temperature range from room temperature to 70° C.

The first artificial breast formation mold was removed. The artificial breast 1 of the present invention at this time point includes the first layer 2 configured by containing the silicone gel without any space, the second layer 3 not covering the entire first layer 2, the second layer 3 being laminated on the outer side part of the first layer 2 on one side, and the third layer 4 directly laminated on the outer side part of the first layer 2 in a state of covering the entire first layer 2 and also laminated on the outer side part of the first layer 2 via the second layer 3.

In addition, the artificial breast 1 of the present invention at this time point further includes the fourth layer 5 not covering the entire third layer 4, the fourth layer 5 being laminated on the outer side part of the third layer 4 on the opposite side of the side where the third layer 4 is in contact with the first layer 2, and the thin skin portion 7 attached to the third layer 4 and provided over the entire peripheral edge portion. Further, the artificial breast 1 of the present invention at this time point includes the polyethylene sheet layer 8 laminated on the substantially flat part of the fourth layer 5 on the opposite side of the third layer 4, the cover layer 62 laminated on the polyethylene sheet layer 8, and the adhesive layer 9 laminated on the polyethylene sheet layer 8 via the cover layer 62.

In addition, when hardness of the first layer 2 immediately after formation of the first layer 2 was measured by a penetrometer, penetration was “55.” The penetrometer is a measuring gauge to be used at the time of measuring hardness of a gel-like substance which is even softer than ultrasoft rubber. In addition, the penetration “55” is substantially the same penetration as firm tofu.

In addition, when the hardness of the first layer 2 was measured again by the penetrometer after about 2 years after the formation of the first layer 2, the hardness was “56” and it was confirmed that its softness was kept.

In addition, when hardness of the second layer 3 immediately after formation of the second layer 3 was measured by a rubber hardness meter, “Type A Durometer,” the hardness was “31.” In addition, when hardness of the third layer 4 immediately after formation of the third layer 4 was measured by the rubber hardness meter, “Type A Durometer,” the hardness was “33” and it was confirmed that the third layer 4 was harder than the second layer 3.

Further, when the artificial breast 1 of the present invention was touched by hand, there were almost no strange feel and unnaturalness peculiar to an artificial breast configured by a plurality of layers. This is thought to be because the second layer 3 and the third layer 4 were in a nearly integrated state.

In addition, even when the obtained artificial breast 1 of the present invention was deformed by applying forces from various angles, unnatural wrinkles were not made.

Next, by using a brush, a colorant was applied to the outer side part of the third layer 4 on the opposite side of the side where the third layer 4 is in contact with the second layer 3. The artificial breast 1 of the present invention to which the colorant was applied was cured within the temperature range from room temperature to 70° C., and the colored layer 61 was formed.

The mixture of the silicone rubber (trade name: VST-50, manufactured by Factor II, Incorporated) and the curing agent (trade name: VST-50-C, manufactured by Factor II, Incorporated) was applied to the colored layer 61 on the opposite side of the third layer 4. After the application, the artificial breast 1 of the present invention was cured within the temperature range from room temperature to 70° C., and the cover layer 62 was formed.

Further, a mixture of an acetoxy dispersing agent containing cyclohexane (product code: MD-564, manufactured by Factor II, Incorporated) and a silicone adhesive (trade name: A-564, manufactured by Factor II, Incorporated) was applied to the cover layer 62 on the opposite side of the colored layer 61. After the application, the artificial breast 1 of the present invention was cured within the temperature range from room temperature to 70° C., and the reflection suppression layer 63 was formed.

The texture of the obtained artificial breast 1 of the present invention becomes closer to the texture of the human skin by the external coloring based on the colored layer 61. In addition, by the cover layer 62, the texture brought close to the texture of the human skin is easily maintained. Further, by the reflection suppression layer 63, the reflection of light on the surface of the artificial breast 1 of the present invention is suppressed, and the texture becomes even closer to the texture of the human skin.

As described above, the artificial breast of the present invention includes the first layer configured by containing silicone gel. Thus, it is possible to keep its softness for a long time in comparison to an artificial breast not configured by containing silicone gel.

In addition, the artificial breast of the present invention includes the first layer configured by containing silicone gel, the second layer arranged partly on the outer side of the first layer, the second layer being harder than the first layer, and the third layer arranged on the outer side of the first layer and the second layer, the third layer being harder than the first layer and the second layer. Thus, the artificial breast of the present invention becomes harder stepwise from the inner side to the outer side, and even when a force is applied from the exterior, unnatural wrinkles are not easily made.

In addition, in order to arrange the third layer on the outer side of the second layer, the silicone rubber of the third layer is applied to the artificial breast formation mold prior to the silicone rubber of the second layer. In the artificial breast of the present invention, since the curing speed of the silicone rubber of the third layer is slower than the curing speed of the silicone rubber of the second layer, it is possible to make the curing of the silicone rubber of the second layer and the curing of the silicone rubber of the third layer occur at substantially the same timing. As a result, it is possible to blur the border line between the second layer and the third layer and bring the second layer and the third layer into a nearly integrated state. Thus, it is possible to reduce unnaturalness upon touching the artificial breast of the present invention.

Therefore, the artificial breast of the present invention is less likely to become unnatural in comparison to a human breast.

In addition, the silicone rubber body of the present invention includes the silicone rubber layer configured by containing silicone rubber. Thus, it is possible to simulate the feel of the human skin.

In addition, the silicone rubber body of the present invention includes the colored layer. This colored layer is arranged on the outer side of the silicone rubber layer and configured by containing the colorant. Thus, it is possible to perform external coloring and bring the texture closer to the texture of the human skin than in the case of internal coloring where silicone rubber contains a colorant.

Further, the silicone rubber body of the present invention includes the cover layer. This cover layer is arranged on the outer side of the colored layer and is capable of suppressing the peeling of the colored layer. Thus, it is possible to maintain the texture brought close to the texture of the human skin.

In addition, the silicone rubber body of the present invention includes the reflection suppression layer. This reflection suppression layer is arranged on the outer side of the cover layer and is capable of suppressing the reflection of light of the cover layer or the colored layer. Thus, it is possible to bring the texture even closer to the texture of the human skin.

Therefore, the silicone rubber body of the present invention is less likely to become unnatural in comparison to the human skin particularly in terms of appearance. As a result, it is possible to apply the silicone rubber body of the present invention to various points of the human body.

### DESCRIPTION OF REFERENCE NUMERALS

1 artificial breast
2 first layer
3 second layer
3A nipple portion
4 third layer
5 fourth layer
6 outer covering layer
61 colored layer
61A nipple region
61Aa papilla region
61Ab areola region
62 cover layer
63 reflection suppression layer
7 thin skin portion
8 polyethylene sheet layer
9 adhesive layer
10 chest portion

1. An artificial breast, comprising:
a first layer configured by containing silicone gel;
a second layer configured by containing silicone rubber and arranged partly on an outer side of the first layer, the second layer being harder than the first layer;
a third layer configured by containing silicone rubber and arranged on the outer side of the first layer and the second layer, the third layer being harder than the first layer and the second layer; and
a fourth layer configured by containing silicone rubber and arranged partly on an outer side of the third layer, the fourth layer having substantially the same hardness as the third layer, wherein
a curing speed of the silicone rubber of the third layer is slower than a curing speed of the silicone rubber of the second layer.

2. The artificial breast according to claim 1, comprising:
a colored layer arranged on the outer side of the third layer, the colored layer being configured by containing a colorant; and
a cover layer arranged on an outer side of the colored layer, the cover layer being capable of suppressing peeling of the colored layer.

3. The artificial breast according to claim 2, wherein the colored layer has a nipple region in which a nipple is displayed, and the nipple region and other regions of the colored layer are positioned in the same plane.

4. The artificial breast according to claim 2, comprising:
a reflection suppression layer arranged on an outer side of the cover layer, the reflection suppression layer being capable of suppressing reflection of light of the cover layer or the colored layer.

5. The artificial breast according to claim 1, comprising:
a thin film layer arranged on an outer side of the fourth layer, the thin film layer being capable of blocking a substance, with the thin film layer being transparent.

6. The artificial breast according to claim 5, comprising:
an adhesive layer arranged attachably to and detachably from a layer which is arranged on the outermost side, the adhesive layer being configured by containing an adhesive material.

7. The artificial breast according to claim 1, wherein the first layer is configured by containing the silicone gel without any space.

8. The artificial breast according to claim 1, wherein the third layer is arranged to cover the entire first layer.

9. A silicone rubber body, comprising:
a silicone rubber layer configured by containing silicone rubber;
a colored layer arranged on an outer side of the silicone rubber layer, the colored layer being configured by containing a colorant;
a cover layer arranged on an outer side of the colored layer, the cover layer being capable of suppressing peeling of the colored layer; and
a reflection suppression layer arranged on an outer side of the cover layer, the reflection suppression layer being capable of suppressing reflection of light of the cover layer or the colored layer.
